(19) United States
(12) Patent Application Publication
(10) Pub. No.: US 2022/0241644 A1
(43) Pub. Date: Aug. 4, 2022

(54) SMART MAT, TERMINAL, SYSTEM AND CONTROL METHOD THEREOF FOR INTERACTIVE TRAINING

(71) Applicant: Omolle Inc., Seoul (KR)

(72) Inventors: Minki KANG, Seoul (KR); Sungho YOO, Seoul (KR); Seongjin HONG, Bucheon-si (KR); Taejun PARK, Seongnam-si (KR); Giyeon NAM, Seoul (KR)

(73) Assignee: Omolle Inc., Seoul (KR)

(21) Appl. No.: 17/507,244

(22) Filed: Oct. 21, 2021

(30) Foreign Application Priority Data
Feb. 1, 2021 (KR) 10-2021-0014328
Mar. 22, 2021 (KR) 10-2021-0036413

(51) Int. Cl. A63B 24/00; A63B 21/00; A63B 71/06; G16H 20/30; G16H 40/67

(52) U.S. Cl. CPC A63B 24/0062; A63B 21/4037; A63B 71/0622; G16H 20/30; G16H 40/67; A63B 2214/00; A63B 2220/836; A63B 2220/40; A63B 2071/063; A63B 2024/0068; A63B 2220/803

(57) ABSTRACT

The present disclosure relates to an interactive fitness system and a control method thereof for detecting an action of a user moving on a smart mat while wearing a wearable terminal, and providing feedback based on the detected result. More specifically, the present disclosure relates to a technology for transmitting and receiving data with at least one of a smart mat and a wearable terminal, and determining whether or not a user is performing training correctly based on the transmitted and received data.

### CROSS-REFERENCE TO RELATED APPLICATION

This application claims priority to Korean Patent Application No. 10-2021-0014328 filed on Feb. 1, 2021 and Korean Patent Application No. 10-2021-0036413 filed on Mar. 22, 2021 and all the benefits accruing therefrom under 35 U.S.C. § 119, the contents of which are incorporated by reference in their entirety.

### BACKGROUND

The present disclosure relates to an apparatus, a system, and a control method for interactive fitness, and more specifically, to an apparatus, a system, and a control method thereof for detecting an action of a user moving on a smart mat while wearing a wearable terminal and providing an optimal feedback based on the detected result.

As restrictions on outdoor activities become severe due to COVID-19, the needs for a home training system is accelerating. The development of such a system is being attempted in various directions, such as a technique for effectively performing an exercise in a narrow space, as well as a technique capable of inducing an active participation of a user.

Korean registered patent No. 10-2141288 (title of invention: method and system for providing home training) provides an action analysis device that analyzes an action of a user based on an angle detected through a wearable terminal by causing the wearable terminal to be worn on a body of the user in order to detect motion of the user who performs an exercise at home. And, the registered patent suggests a configuration that induces active participation of the user by providing feedback on the analyzed action.

However, due to a fact that a separate device for analyzing the action of the user is required according to the registered patent, there is a problem that a separate installation space for the device is required as well as a problem that installation and maintenance costs of the system may increase.

Therefore, there is a need for research on a device and system that can not only induce active participation through analysis of the action of the user during home training, but also lower the installation cost and enables efficient use of space.

### SUMMARY

The present disclosure provides a smart mat, an interactive fitness device, a system, and a control method thereof for home training.

The present disclosure also provides an interactive fitness system for accurately detecting a training motion action of a user and providing feedback without a separately installed device.

Embodiments to be implemented in the present disclosure are not limited to the embodiments mentioned above, and other embodiments not mentioned will be clearly understood by those of ordinary skill in the art to which the present disclosure belongs from the following description.

In accordance with an exemplary embodiment of the present invention, an interactive fitness terminal includes: a memory configured to store instructions; a communication unit configured to transmit and receive data with at least one of a smart mat and a wearable terminal; and a control unit set to execute the stored instructions, in which the control unit determines whether or not a user is performing training correctly based on data received through the communication unit.

The smart mat may be configured to acquire contact data, which is data obtained by detecting contact with at least a part of a body of the user.

The wearable terminal may be configured to be worn on a part of the body of the user to acquire motion data, which is data obtained by detecting a motion of the part of the user's body.

The control unit may be configured to control the communication unit so as to receive at least one of the contact data and the motion data, and make the determination based on at least one of the received contact data and motion data.

The control unit may be configured to output the determined result.

In accordance with another exemplary embodiment of the present invention, a control method of an interactive fitness terminal includes: receiving data from at least one of a smart mat and a wearable terminal; and determining whether or not a user is performing training correctly based on the received data.

The smart mat may be configured to acquire contact data, which is data obtained by detecting contact with at least a part of a body of a user.

The wearable terminal may be configured to be worn on a part of the body of the user to acquire motion data, which is data obtained by detecting a motion of the part of the user's body.

In the receiving, at least one of the contact data and the motion data may be received, and the determination may be made based on at least one of the received contact data and motion data.

The control method may further includes outputting the determined result.

Additional scope of applicability of the present disclosure will become obvious from the following detailed description. However, since various alterations and modifications within the technical spirit and scope of the present disclosure can be clearly understood by those skilled in the art to which the present disclosure belongs, it should be understood that the specific embodiments described in the detailed description are given by way of example only.

### BRIEF DESCRIPTION OF THE DRAWINGS

FIG. 1 illustrates a conceptual diagram of an interactive fitness system 100 in accordance with exemplary embodiment of the present invention.

FIG. 2 is a conceptual diagram for describing an interactive action in accordance with an exemplary embodiment of the present invention.

FIG. 3 illustrates a training screen of a user terminal 130 for implementing the interactive action in accordance with another exemplary embodiment of the present invention.

FIG. 4 illustrates a block diagram of a smart mat 110 in accordance with still another exemplary embodiment of the present invention.

FIG. 5 illustrates a block diagram of a wearable terminal 120 and the user terminal 130 in accordance with still another exemplary embodiment of the present invention.

FIG. 6 illustrates a block diagram of a server 140 in accordance with still another exemplary embodiment of the present invention.

FIG. 7 is a diagram illustrating a control sequence of the interactive fitness system 100 in accordance with still another exemplary embodiment of the present invention.

FIG. 8 is a diagram for describing a specific method of judging an exercise action in accordance with still another exemplary embodiment of the present invention.

FIG. 9 illustrates a conceptual diagram for considering action sync judgment and continuity (continuity judgment) between detected actions in accordance with still another exemplary embodiment of the present invention.

FIG. 10 illustrates a conceptual diagram for calculating calorie consumption, exercise points, and ranking in accordance with still another exemplary embodiment of the present invention.

FIG. 11 is a diagram illustrating an example of a feedback voice in accordance with still another exemplary embodiment of the present invention.

### DETAILED DESCRIPTION OF EMBODIMENTS

Hereinafter, the embodiments disclosed in the present specification will be described in detail with reference to the accompanying drawings, but the same or similar components are assigned the same reference numerals regardless of reference symbols, and redundant descriptions thereof will be omitted. The suffixes “module” and “unit” for components used in the following description are given or mixed in consideration of only the ease of writing the specification, and do not have distinct meanings or roles by themselves. In addition, in describing the embodiments disclosed in the present specification, if it is determined that a detailed description of a related known technology may obscure the gist of the embodiments disclosed in the present specification, the detailed description thereof will be omitted. In addition, the accompanying drawings are only for easy understanding of the embodiments disclosed in the present specification, and the technical ideas disclosed in the present specification are not limited by the accompanying drawings, and it should be understood that the accompanying drawings include all alterations, equivalents, and substitutions included in the spirit and technical scope of the present invention.

Terms including ordinal numbers such as first, second, etc. may be used to describe various components, but the components are not limited by the terms. The above terms are used only for the purpose of distinguishing one component from other components.

When it is mentioned that a certain component is “coupled” or “connected” to another component, it should be understood that the component may be directly coupled to or connected to the other component, but other component may exist therebetween. In contrast, when it is mentioned that a certain element is “directly coupled” or “directly connected” to another element, it should be understood that no other element exists therebetween.

The singular expression includes the plural expression unless the context clearly dictates otherwise.

In the present application, terms such as “comprise” or “have” are intended to designate that a feature, number, step, action, component, part, or combination thereof described in the specification is present, and it should be understood that the terms do not preclude the possibility of addition or existence of one or more other features or numbers, steps, actions, components, parts, or combinations thereof.

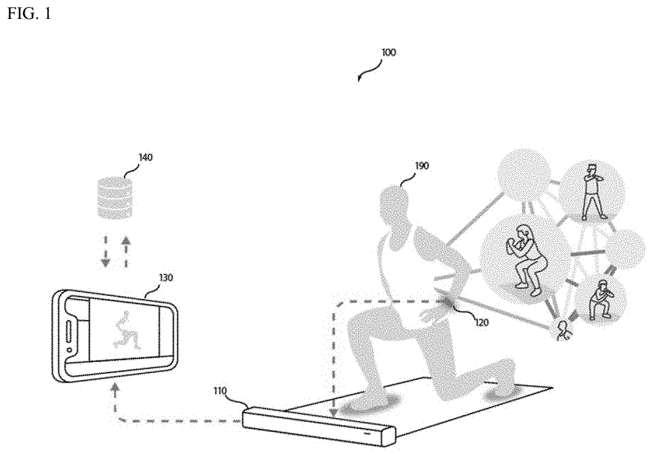

FIG. 1 illustrates a conceptual diagram of the interactive fitness system 100 in accordance with exemplary embodiment of the present invention.

The interactive fitness system 100 according to an embodiment of the present disclosure can be configured to include a smart mat 110, a wearable terminal 120, a user terminal 130, and a server 140. The components illustrated in FIG. 1 are not essential in implementing the interactive fitness system 100, and thus the interactive fitness system 100 described in this specification can include more or fewer components than those listed above.

The smart mat 110 is provided to acquire contact data, which is data obtained by detecting contact with at least a part of a body of a user 190. As a representative example, the smart mat 110 can be generally provided in the form of a yoga mat used at home or a fitness center and detect that a part of the body, such as a sole, a knee, a hand, or a hip comes into contact with the smart mat 110 when the user 190 performs various actions on the smart mat 110. It may be obvious that the smart mat 110 can detect a plurality of contact points at the same time.

The wearable terminal 120 is configured to be worn on a part of a body of the user 190 and to acquire motion data, which is data obtained by detecting motion of the part of the body. The wearable terminal 120 according to an embodiment of the present disclosure can be provided in the form of a watch or a band worn on the wrist of the user 190. As an example, the wearable terminal 120 is a smart watch (e.g., a Galaxy watch or an Apple watch), and embodiments of the present disclosure may be implemented through sensors provided in the smart watch.

Hereinafter, the contact data and the motion data are collectively referred to as action data.

As illustrated in the drawing, the motion data detected by the wearable terminal 120 may be transmitted to the user terminal 130 through the smart mat 110, but may not necessarily be limited thereto. That is, the wearable terminal 120 may exchange data with the smart mat 110 as illustrated in the drawing, but may exchange data with the user terminal 130, or the wearable terminal 120 may be directly connected to an online/offline network to exchange data with the server 140.

The user terminal 130 is a configuration unit for receiving data for analyzing behavior of the user 190 from the smart mat 110 and the wearable terminal 120 and transmitting the received data to the server 140 for analysis, or directly performing analysis. In addition, the user terminal 130 according to an embodiment of the present disclosure can output a training video image to the user 190. Such a training video image may be provided in a form of real-time streaming but a previously recorded and stored video image may be provided. Such a training video image may be output in the form stored in the user terminal 130, but may be output in a form in which the training video image stored in a video image database (to be described below) of the server 140 is provided to the user terminal 130.

According to one embodiment of the present disclosure, the user terminal 130 may correspond to at least one of a mobile phone, a cellular phone, a smart phone, a personal computer, a laptop, a notebook computer, a netbook or tablet, a personal digital assistant (PDA), a digital camera, a game console, an MP3 player, a personal multimedia player (PMP), an electronic book (E-Book), a navigation device, a disk player, a set-top box, a home appliance, a communication device, and a display device.

The server 140 is a configuration unit for exchanging data with the user terminal 130. As an example, the server 140 can receive action data from the user terminal 130 and analyze an action of the user 190.

Home training has a clear advantage of saving time and money, but there is a problem in that it is difficult to induce active participation.

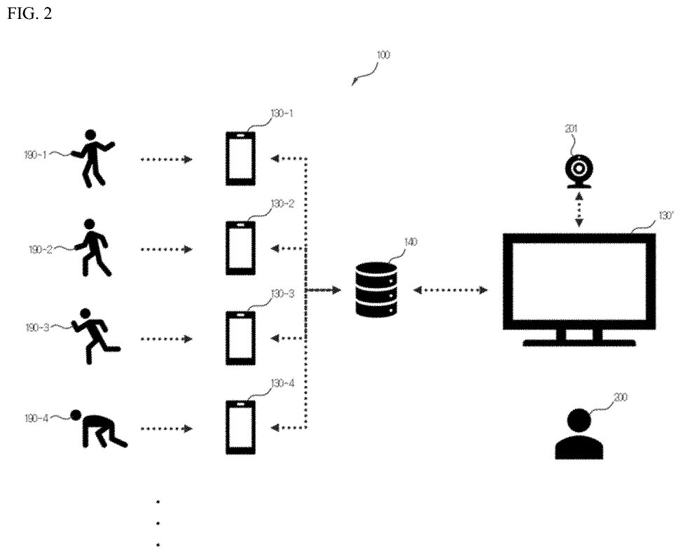

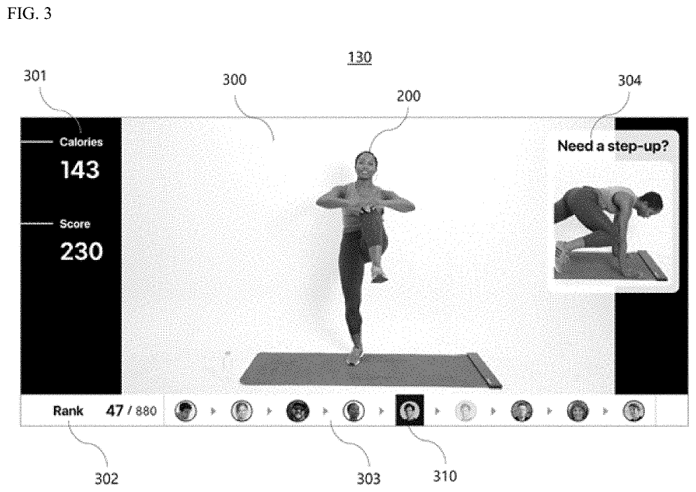

In the present disclosure, in order to induce active participation of the user, a system, in which the user can exercise not only with a trainer but also with other users in a virtual online space even when the user exercises alone at home, is suggested. Through this, the user can participate in the exercise more actively, and the effect of the exercise can be further improved by providing an encouraging feedback to the users who are lagging behind and inducing competition with other users. To this end, the server 140 according to an embodiment of the present disclosure can be provided to exchange data with a plurality of user terminals 130. The concept of interactive will be described in more detail with reference to FIGS. 2 and 3.

FIG. 2 is a conceptual diagram for describing an interactive action in accordance with an exemplary embodiment of the present invention. FIG. 3 illustrates a training screen of the user terminal 130 for implementing the interactive action in accordance with another exemplary embodiment of the present invention. Description will be made with reference to FIGS. 2 and 3 together. In the illustrated drawings, a system including a plurality of user terminals 130-1 to 130-4 is described, and when an individual operation for each of the plurality of user terminals 130-1 to 130-4 is described, the user terminals 130-1 to 130-4 will be referred to as the user terminal 130.

As illustrated in the drawing, the interactive fitness system 100 according to an embodiment of the present disclosure provides a system in which a plurality of users 190-1 to 190-4 can perform home training together. Each of the plurality of users 190-1 to 190-4 can exchange data with the server 140 through each of their user terminals 130-1 to 130-4.

According to an embodiment of the present disclosure, the server 140 can provide a training video image 300 to the plurality of user terminals 130-1 to 130-4. In this case, as described above, the training video image 300 to be provided may be provided in a form of real-time streaming, or a previously recorded and stored image may be provided.

Although the plurality of users 190-1 to 190-4 perform training in their respective homes, each user can expect an effect of participating more actively than when performing the training alone by feeling as if they are training together while viewing the same training video image 300.

In this case, each user 190 can perform training according to the training video image 300 being output while wearing the wearable terminal 120 on his/her smart mat 110. The user terminal 130 may receive action data acquired by the smart mat 110 and the wearable terminal 120 and transmit the action data to the server 140.

When the server 140 receives the action data, the server 140 can check whether or not the user 190 is correctly performing training based on the action data. In addition, the server 140 can provide the user terminal 130 with calorie data obtained by calculating calorie consumption based on the received action data. Based on the calorie data, the user terminal 130 can output calorie consumption information 301. Depending on the embodiment, a point can be output together.

The server 140 can determine the ranking of each user based on the action data received from the plurality of user terminals 130-1 to 130-4, and provide ranking information therefor to each of the plurality of user terminals 130-1 to 130-4. Each user terminal 130 that has received the ranking information can output current ranking information 302 and a real-time ranking display bar 303. As in the illustrated example, on the time ranking display bar 303, the ranking information of other users 190 currently viewing the same training video image 300 is displayed together, and the ranking can be provided in the form of a profile image 310.

According to another embodiment, the user terminal 130 may provide a suggestion video image 304 based on the received ranking information or point information. When ranking information or point information of the user is lower than the reference value, the suggestion video image 304 may be a video image that suggests an easier exercise than the training video image 300 currently being output. Alternatively, when the ranking information or point information of the user is higher than a reference value, the suggestion video image 304 may be a video image that suggests an exercise of higher difficulty than the training video image 300 currently being output.

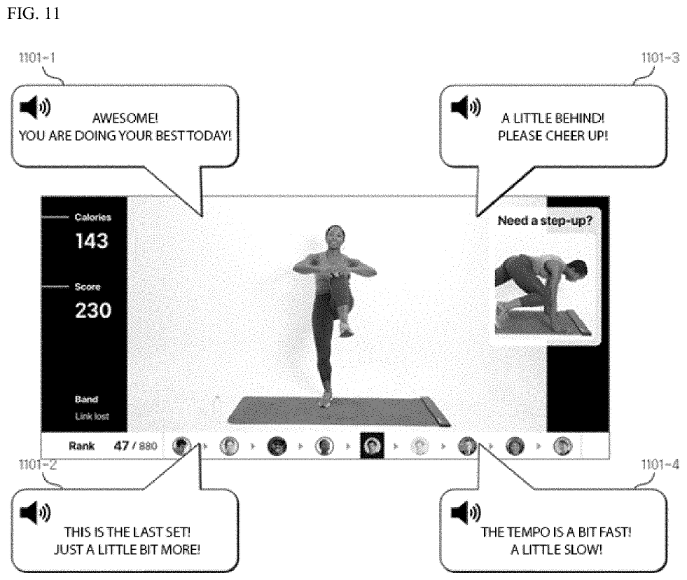

Furthermore, the user terminal 130 according to an embodiment of the present disclosure can output a feedback voice based on the received ranking information or point information. An example of such a feedback voice will be described later with reference to FIG. 11.

When the training video image 300 is provided in the form of real-time streaming, a trainer 200 can capture the training video image 300 in real time through the camera 201, and provide the captured training image 300 to the server 140.

The server 140 according to an embodiment of the present disclosure, based on the action data received from the plurality of user terminals 130-1 to 130-4, can monitor whether or not the users are performing correctly in real time and can provide a real-time monitoring screen for outputting the monitoring result through a user terminal 130′ of the trainer 200 (hereinafter referred to as a trainer user terminal). The trainer 200 refers to the real-time monitoring screen and delivers praise feedback if there is a user who is performing well in training among the plurality of users 190-1 to 190-4, or points out an incorrect posture or delivers encouraging feedback if there a user who does not follow the training correctly, thereby capable of inducing active participation. Such feedback of the trainer 200 may be transmitted to each user terminal 130 through real-time streaming.

In the illustrated example, the camera 201 is connected to the trainer terminal 130′ to perform photographing for a real-time streaming video image, but is not necessarily limited thereto, and the camera 201 may provide a streaming video image directly to the server 140 without using the trainer terminal 130′.

In the illustrated drawings, although the embodiment in which the training image 300 is provided in the form of real-time streaming has been described, it is obvious that the same can be applied to the embodiment in which the previously recorded image stored in the image DB of the server 140 is provided.

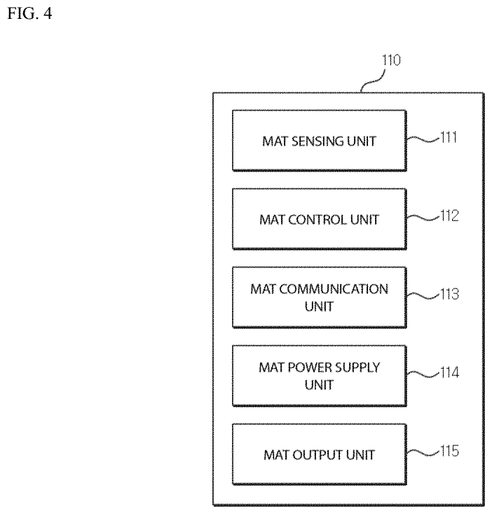

FIG. 4 illustrates a block diagram of the smart mat 110 in accordance with still another exemplary embodiment of the present invention.

The smart mat 110 can include a mat sensing unit 111, a mat control unit 112, a mat communication unit 113, a mat power supply unit 114, a mat output unit 115, etc. The components illustrated in FIG. 4 are not essential in implementing the smart mat 110, and thus the smart mat 110 described in this specification may include more or fewer components than the components listed above.

The mat sensing unit 111 detects contact with at least a part of the body of the user 190. The mat sensing unit 111 detects that the sole, knee, hand, and hip contact the smart mat 110 while the user 190 is training on the smart mat 110. For example, the mat sensing unit 111 may detect this contact through a pressure sensing method. The mat sensing unit 111 according to an embodiment of the present disclosure may detect a contact position and area.

The mat control unit 112 controls the overall operation of the smart mat 110. The mat control unit 112 may judge whether or not the user 190 is performing training correctly based on the action data. It is only an example that such judgment is made on the smart mat 110, and the judgment may be made by at least one of the smart mat 110, the user terminal 130, and the server 140.

The mat communication unit 113 is a configuration unit for exchanging data with at least one of the wearable terminal 120, the user terminal 130, and the server 140.

The mat power supply unit 114 supplies power to each component included in the smart mat 110 by receiving external power and internal power under the control of the mat control unit 112. The mat power supply 114 includes a battery, and the battery may be a built-in battery or a replaceable battery.

The mat output unit 115 can output the judgment result as to whether or not the user 190 is performing training correctly. As described above, the judgment output through the mat output unit 115 may be judgment made by the mat control unit 112, but may also be judgment made by the server 140 or other terminals 120 and 130. For example, a feedback voice of a form to be described later in FIG. 11 can be output through the mat output unit 115.

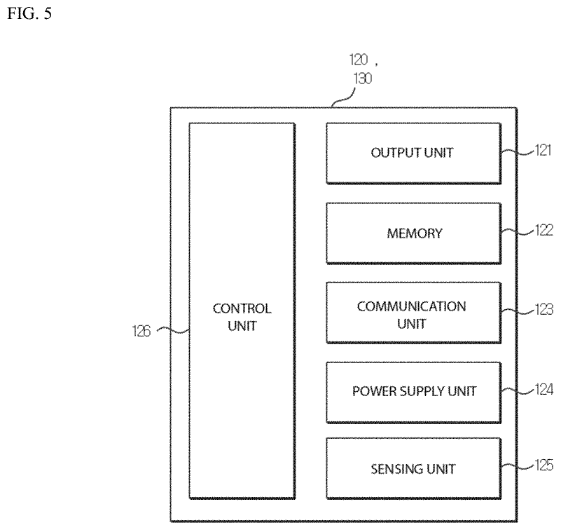

FIG. 5 illustrates a block diagram of the wearable terminal 120 and the user terminal 130 in accordance with still another exemplary embodiment of the present invention. In FIG. 5 and the following detailed description, the wearable terminal 120 and the user terminal 130 are collectively referred to as terminals 120 and 130.

The terminals 120 and 130 can include an output unit 121, a memory 122, a communication unit 123, a power supply unit 124, a sensing unit 125, and a control unit 126. The components illustrated in FIG. 5 are not essential in implementing the terminals 120 and 130, and thus the terminals 120 and 130 described in this specification can include more or fewer components than those listed above.

The output unit 121 means a configuration unit for generating various outputs provided to the user 190, and can include at least one of a display and a sound output unit. The output unit 121 can output appropriate feedback to the user 190 to improve a degree of training participation of the user 190. An example of such feedback will be described later with reference to FIG. 11.

The memory 122 stores data supporting various functions of the terminals 120 and 130. The memory 122 can store data and instructions for the operation of the terminals 120 and 130.

The communication unit 123 can include one or more modules that enable communication between the terminals 120 and 130 and a communication system, between the terminals 120 and 130 and other terminals 120 and 130, or between the terminals 120 and 130 and the external server 140. In addition, the communication unit 123 can include one or more modules for connecting the terminals 120 and 130 to one or more networks.

The power supply unit 124 supplies power to each component included in the terminals 120 and 130 by receiving external power and internal power under the control of the control unit 126. The power supply unit 124 includes a battery, and the battery may be a built-in battery or a replaceable battery.

The sensing unit 125 can include one or more sensors for sensing information in the terminals 120 and 130 and surrounding environment information surrounding the terminals 120 and 130. For example, the sensing unit 125 can include at least one of an acceleration sensor, a gravity sensor (G-sensor), a gyroscope sensor, and a motion sensor. In particular, the wearable terminal 120 disclosed in this specification may acquire motion data of the user 190 by combining and utilizing information sensed by at least two or more of these sensors.

The control unit 126 executes instructions stored in the memory 122 to generally control the overall operation of the terminals 120 and 130. The control unit 126 can provide appropriate information or functions to the user or process the information or functions by processing signals, data, information, etc. input or output through the components described above.

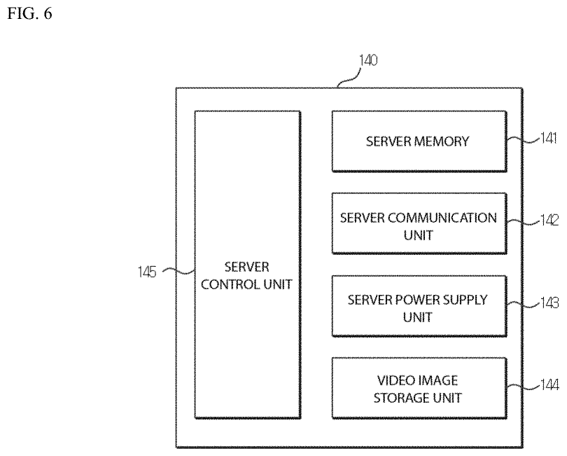

FIG. 6 illustrates a block diagram of the server 140 in accordance with still another exemplary embodiment of the present invention. The server 140 can include a server memory 141, a server communication unit 142, a server power supply unit 143, a video image storage unit 144, and a server control unit 145. The components illustrated in FIG. 6 are not essential for implementing the server 140, and thus the server 140 described in this specification may include more or fewer components than those listed above.

The server memory 141 stores data supporting various functions of the server 140. The server memory 141 can store data and instructions for the operation of the server 140.

The server communication unit 142 can include one or more modules that enable communication between the server 140 and the communication system or between the server 140 and another server 140. In addition, the server communication unit 142 can include one or more modules for connecting the server 140 to one or more networks.

The server power supply unit 143 supplies power to each component included in the server 140 by receiving external power and internal power under the control of the server control unit 145.

The video image storage unit 144 stores a plurality of training video images 300 to be provided to the user terminal 130. When a request for a video image is received from the user terminal 130, the server controller 145 can transmit a training video image 300 corresponding to the request from among the plurality of training video images 300 stored in the video image storage unit 144 to the user terminal 130 as a reply. Furthermore, the video image storage unit 144 can store judgment criterion data corresponding to each of the plurality of training video images 300 together.

The server control unit 145 executes instructions stored in the server memory 141 to generally control the overall operation of the server 140. The server control unit 145 can provide appropriate information or functions to the user or process the information or functions by processing signals, data, information, etc. input or output through the components described above.

In particular, the server control unit 145 according to an embodiment of the present disclosure can determine whether or not the user 190 is correctly performing training, based on the action data received from the terminals 120 and 130.

Hereinafter, a control sequence of the interactive fitness system 100 according to an embodiment of the present invention will be described in more detail with reference to the drawings.

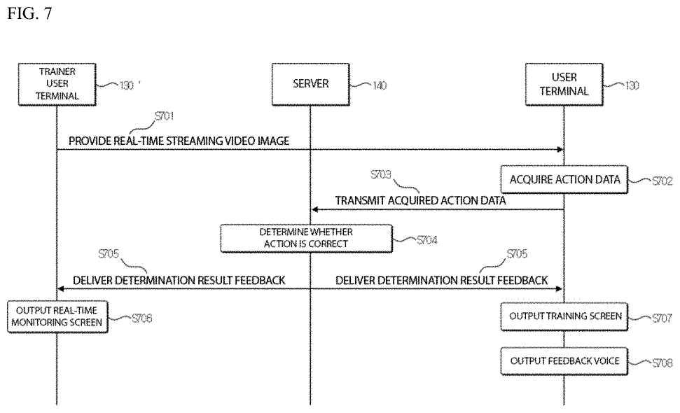

FIG. 7 is a diagram illustrating a control sequence of the interactive fitness system 100 in accordance with still another exemplary embodiment of the present invention. In the illustrated drawing, the smart mat 110 and the wearable terminal 120 are omitted for convenience of description.

First, in step S701, the trainer user terminal 130′ can provide the training video image 300 to the user terminal 130 in the form of real-time streaming. In this case, the training video image 300 to be provided may be provided to the user terminal 130 through the server 140, but may also be provided to the user terminal 130′ without using the server 140. As an example, a real-time streaming video image may be provided through an external real-time streaming platform (e.g., YouTube, etc.).

In addition, as described above, the training video image 300 captured by the camera 201 may be provided through the trainer user terminal 130′, but may be provided without using the trainer user terminal 130′.

Additionally, step S701 may not be performed once due to the characteristics of the streaming form, but may be continuously performed while the training video image is provided.

The user terminal 130 acquires action data in step S702. More specifically, the user terminal 130 can receive contact data from the smart mat 110 and can receive motion data from the wearable terminal 120. The action data means data including both the motion data and the contact data.

The interactive fitness system 100 according to an embodiment of the present disclosure determines whether or not the user 190 is correctly performing the exercise as well as calorie consumption and ranking of the user 190 based on this action data (hereinafter referred to as exercise action judgment). In the example illustrated in FIG. 7, the server 140 is illustrated as making such a determination, but is not necessarily limited thereto, and it is obvious that this determination may be made by at least one of the user terminal 130, the smart mat 110, and the wearable terminal 120. In particular, such a determination is not entirely made by any one entity, but may be performed by different entities, such as calculating calorie consumption by the server 140, performing exercise action judgement by the user terminal 130.

A specific method of judging the exercise action will be described later in detail with reference to the drawings below.

In step S705, the server 140 may deliver feedback of the determination result. In this case, it is obvious that feedback can be delivered not only to the user terminal 130 but also to the trainer user terminal 130′.

The trainer user terminal 130′ can output a real-time monitoring screen based on the feedback in step S705, in S706.

And, the user terminal 130 can output the training screen 300 based on the video image provided in step S701 and the feedback in step S705, in S707. Furthermore, in one embodiment of the present disclosure, it is suggested that the user terminal 130 outputs a feedback voice based on the determination result feedback in S705, in S708. A specific embodiment of such a feedback voice will be described later with reference to FIG. 11.

Steps S701 to S707 described above are not steps performed once, but steps that can be repeatedly performed while the user 190 is training, and the sequence in which the steps is performed may be different from the sequence in the illustrated drawing.

In addition, according to the flowchart illustrated in FIG. 7, the embodiment in which the training video image 300 is provided in the form of real-time streaming has been described, but is not limited thereto, and It may be obvious that the same steps may be performed in an embodiment in which the previously recorded and stored training video image 300 is provided.

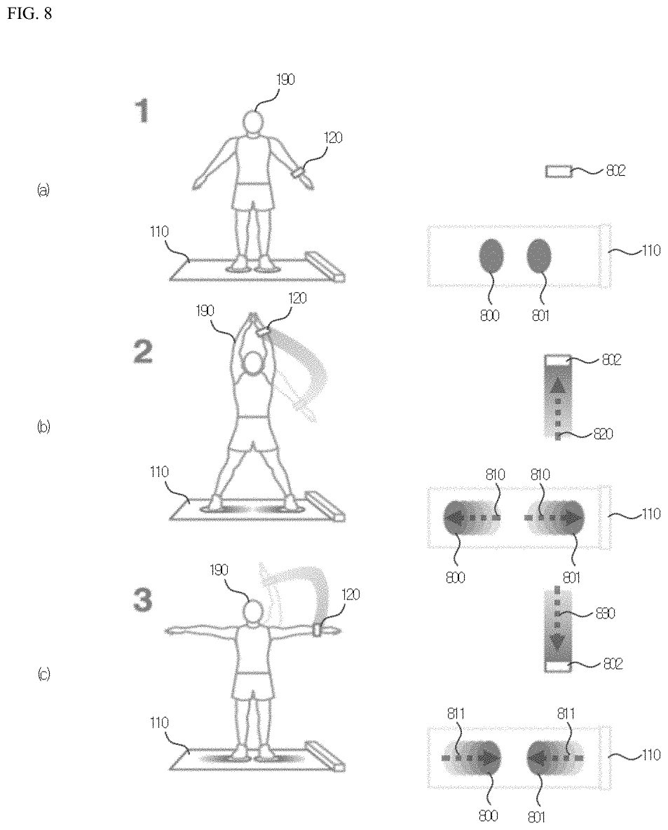

Hereinafter, a specific method of judging an exercise action will be described with reference to FIG. 8. In FIG. 8, an example in which only motion in the vertical direction, with gravity as a reference, on the basis of an acceleration sensor is considered will be described, but is not necessarily limited thereto, and the exercise action judgment in consideration of the motion in a three-dimensional space on the basis of a gyro sensor or other motion sensor may also be included in the present disclosure.

FIG. 8 is a diagram for describing a specific method of judging an exercise action in accordance with still another exemplary embodiment of the present invention. In the illustrated example, a change in contact data and motion data when switching between the respective actions in a case where the user 190 performs actions for an open-arm jumping exercise (performs repetitive exercise in an action sequence of (a)->(b)->(c)->(a)) is described.

First, in action (a), the user 190 waits with both feet about shoulder width apart and both arms lightly spread out. In action (a), the contact data acquired through the smart mat 110 may include first contact data 800 obtained by detecting the right sole and second contact data 801 obtained by detecting the left sole. In this case, a relative position between the first and second contact data 800 and 801 may be maintained at an appropriate interval (about a shoulder width). In addition, motion data acquired through the wearable terminal 120 in action (a) will include motion data 802 in which the height is kept constant. That is, in action (a) itself, a change in height may not be detected.

The (b) action is a follow-up action to action (a), and includes a lower body action of the user 190 to widen the distance between the feet while jumping lightly and an upper body action with arms clasped above head.

First, in the case of analyzing the lower body action, when switching from action (a) to action (b), the first and second contact data 800 and 801 will move in a direction 810 away from each other. The server 140 detects a first lower body movement change by determining whether or not the first and second contact data 800 and 801 move apart when switching from action (a) to action (b). In this case, the server 140 may determine whether or not the distance at which the first and second contact data 800 and 801 move away from each other is greater than a preset value, and determine whether or not the first lower body action switching has been correctly performed. If the first lower body action switching is not correctly performed, the server 140 may not detect that the first lower body action switching has been performed.

Then, in the case of analyzing the upper body action, when switching from action (a) to action (b), the motion data will include data moving in a direction 820 (opposite to the direction of gravity) higher than the height in action (a). Accordingly, the server 140 detects first upper body action switching by determining whether or not the motion data moves in a direction in which the position becomes higher than the height in action (a) when switching from action (a) to action (b). Similarly, the server 140 may determine whether or not the change in height is greater than a preset value, and determine whether or not the first upper body action switching is correctly performed. If the change in height is smaller than the preset value, the server 140 may not detect that the first upper body action switching has been performed.

The server 140 may perform exercise action judgment based on whether or not the first lower body action switching and the first upper body action switching are correctly detected.

Subsequently, the detection of a second upper body action switching and a second lower body action switching when switching from action (b) to action (c) will be described.

In the case of analyzing the lower body action, when switching from action (b) to action (c), the first and second contact data 800 and 801 will move in a direction 811 in which they come close to each other. The server 140 detects the second lower body action switching by determining whether or not the first and second contact data 800 and 801 come close to each other when switching from action (b) to action (c). In this case, the server 140 may determine whether or not a distance at which the first and second contact data 800 and 801 come close to each other is greater than a preset value, and determine whether or not the second lower body action switching has been correctly performed. If the second lower body action switching is not correctly performed, the server 140 may not detect that the second lower body motion change has been performed.

In the case of analyzing the upper body action, when switching from action (b) to action (c), the motion data will include data moving in a direction 830 (the direction of gravity) lower than the height in action (b). Accordingly, the server 140 detects second upper body action switching by determining whether or not the motion data moves in a direction in which the position becomes lower than the height in action (b) when switching from action (b) to action (c). Similarly, the server 140 may determine whether or not the change in height is greater than a preset value, and determine whether or not the second upper body action switching is correctly performed. If the change in height is smaller than the preset value, the server 140 may not detect that the second upper body action switching has been performed.

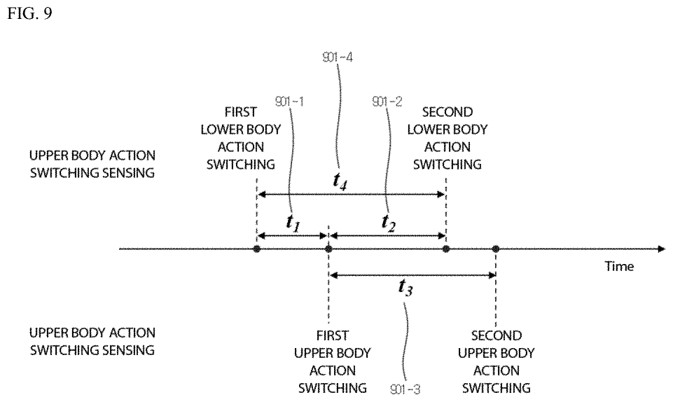

In performing exercise action judgment, the system 100 according to an embodiment of the present disclosure suggests to make a determination (hereinafter referred to as action sync judgment) as to whether the synchronization between the upper body action switching and the lower body action switching is established together, rather than simply discriminately judge the upper body action and the lower body action. That is, it is suggested to determine by combining contact data and motion data together. Hereinafter, description will be made with reference to FIG. 9 together.

FIG. 9 illustrates a conceptual diagram for considering action sync judgment and continuity (continuity judgment) between detected actions in accordance with an exemplary embodiment of the present invention. In FIG. 9, the time axis is illustrated, and the time points of the upper and lower body actions detected over time are indicated.

For example, when switching from (a) action to (b) action in an open-arm jumping exercise, it will be common that the lower body action and the upper body action are done together. This is because, when the lower body exercise of spreading the legs is done, the upper body exercise of raising the arms above the head is done. Accordingly, the action sync judgment described above may be performed based on whether or not a time point at which the first lower body action switching is detected and a time point at which the first upper body action switching is detected are within or outside a predetermined interval.

Accordingly, the server 140 may calculate a first time difference t₁, 901-1 between the time point when the first lower body action switching is detected and the time point when the first upper body action switching is detected, determine whether or not the first time difference is smaller than a predetermined value, and perform the action sync judgement. Alternatively, the action sync judgment may be performed according to whether the first time difference is greater than the predetermined value or within the predetermined range according to the type of exercise to be performed.

Furthermore, the server 140 according to an embodiment of the present disclosure may consider a time difference between a plurality of upper and lower body actions together when performing the sync judgment. This is because, in the case of a simple exercise such as the open-arm jumping exercise in the example described above, the upper body action and lower body action may be matched 1:1, but in the case of a complex exercise, several upper and lower body actions may be synchronized with each other.

That is, the server 140, with the first upper body action switching as a reference, may perform the action sync judgment by considering not only the first time difference t₁, 901-1 with the first lower body action switching but also the second time difference t₂, 901-2 with the second lower body action switching.

Furthermore, the system 100 according to an embodiment of the present disclosure suggests to further consider the continuity between the detected actions in performing the exercise action judgment. This is because the synchronization between upper and lower body actions is important, but switching between upper and lower body actions can also be important.

To this end, the server 140 according to an embodiment of the present disclosure suggests to consider a third time difference t₃, 901-3 between the detected upper body actions or a fourth time difference t₄, 901-4 between the 1 detected lower body actions.

When switching from the first upper body action switching to the second upper body action switching is made too soon or too late than the normal case, it will not be considered that the correct exercise has been performed. Accordingly, the server 140 according to an embodiment of the present disclosure suggests to perform the continuity judgment based on whether or not the third time difference t₃, 901-3 or the fourth time difference t₄, 901-4 falls within a predetermined range so as not to be too small or too large.

According to the description in FIGS. 8 and 9, the server 140 according to an embodiment of the present disclosure made the determination based on a pattern of a change in contact data and a change in motion data corresponding to the open-arm jumping exercise. Exercise action judgment for other exercises, without being limited to the exercise, may be performed with a pattern for a change in contact data a change in motion data for various types of exercises as a reference. Accordingly, in one embodiment of the present disclosure, it is suggested to store a pattern of a change in contact data and a change in movement data, which is a criterion for judging an exercise action for each exercise, and the pattern of a change in contact data and a change in motion data, which is the criterion for judgment, will be referred to as ‘judgment criterion data’.

The judgment criterion data may include a criterion for contact data or motion data for determining a specific action (actions (a) to (c) in the example of FIG. 8), and may include a criterion for a change in contact data or motion data for detecting switching between actions. Furthermore, the judgment criterion data may also include information on a criterion of a time difference between action switching for the action sync judgment and information on a time difference criterion between successive action switching for continuity judgment.

Each judgment criterion data corresponding to each of a plurality of actions may be stored in at least one of the smart mat 110, the terminals 120 and 130, and the server 140, and judgment criterion data corresponding to the training video image 300 reproduced in the user terminal 130 may be loaded and used to judge an exercise action. That is, when the training video image 300 includes an action for ‘exercise A’, the server 140 may load judgment criterion data for ‘exercise A’ from among the stored judgment criterion data and perform the exercise action judgment for ‘exercise A’.

Meanwhile, in the embodiment described above, an example in which the judgments are performed by the server 140 has been described, but is not necessarily limited thereto, and the judgments may be performed by the terminals 120 and 130 as described above. If the judgment is made by the terminals 120 and 130, It may be obvious that the terminals 120 and 130 can load the judgment criterion data for the exercise action judgment.

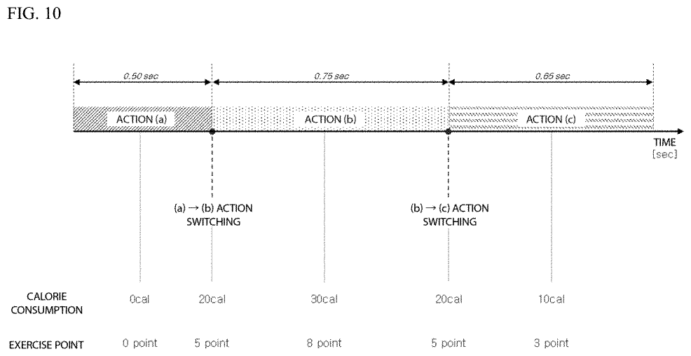

Meanwhile, at least one of the smart mat 110, the terminals 120, 130, and the server 140 may calculate at least one of calories consumed by training, exercise points, and ranking based on the exercise action judgment described above. A specific calculation method will be described below with reference to FIG. 10.

FIG. 10 illustrates a conceptual diagram for calculating calorie consumption, exercise points, and ranking in accordance with still another exemplary embodiment of the present invention. The interactive fitness system 100 according to an embodiment of the present disclosure suggests to calculate the calorie consumption according to the detected action and switching of the action.

Referring to the example in FIG. 10, actions (a) to (c) are sequentially switched while action (a) is maintained for approximately 0.50 seconds, action (b) is maintained for approximately 0.75 seconds, and action (c) is maintained for approximately 0.65 seconds.

In one embodiment of the present disclosure, it is suggested to calculate not only the calories consumed for each action, but also the calories consumed when each action is switched. This is to calculate more accurate calorie consumption.

Table 1 is an example of a calorie calculation table according to an embodiment of the present disclosure.

TABLE 1

| Item | Calorie consumption | | | Remarks |
|---|---|---|---|---|
| action (a) | 0 | | | standby motion and no |
| | | | | calorie consumption |
| (a) → (b) | within | 0.5 to 1.0 sec | 1.0 sec | when switching between |
| action | 0.5 sec | | or more | actions is fast, calorie |
| switching | 20 cal | 17 cal | 15 cal | consumption is high |
| action (b) | 40 cal/s | | | consume calories in |
| | | | | proportion to holding time |
| (b) → (c) | within | 0.5 to 1.0 sec | 1.0 sec | when switching between |
| action | 0.5 sec | | or more | actions is fast, calorie |
| switching | 27 cal | 25 cal | 23 cal | consumption is high |

| | | |
|---|---|---|
| action (c) | 10 cal | consume fixed calories |
| | | regardless of holding time |

The calculation method for each item may be different. For example, in the case of action (a), it is a simple standby operation, and there may be no calorie consumption. In addition, in the case of action (c), calorie consumption may be calculated regardless of a time period during which the action is maintained.

In the case of an item in which the action is switched from action (a) to action (b), more calorie consumption is expected if a switching speed is fast, and calorie consumption will be less if the switching speed is slow. Accordingly, the calorie consumption in the item in which the action is switched from action (a) to action (b) may vary depending on a time period during which action (a) is maintained. For example, when action (a) is maintained for approximately 0.50 seconds and switched to action (b) as illustrated in the drawing, approximately 20 cal may be consumed.

In the case of a specific action, even if the user is not particularly moving, there may be calorie consumption in maintaining the specific action itself. In the case of the item including such an action, calories consumed in proportion to the time period during which the corresponding action is maintained may be calculated. For example, in the case of (b) action, approximately 40 cal is consumed per second, and thus in the case of (b) action that is maintained for approximately 0.75 seconds, it is possible to estimate the calories consumed by approximately 40*0.75=30 cal.

Additionally, in the case of the calculation table according to Table 1, it may be calculated so that calories are consumed only when it is determined that the action is correct in step S704. For example, when (b) action is correctly detected but (c) action is not judged to be a correct action, it is possible to calculate the calories consumed by excluding calorie consumption for action (c).

The interactive fitness system 100 according to an embodiment of the present disclosure suggest to further calculate the exercise points acquired by the user 190, together with the calculation of calorie consumed. The exercise points mean that the result of the action of the user 190 is digitized, and when it is recognized that the user 190 has performed the correct operation, the exercise points may be provided to the user 190.

Furthermore, the interactive fitness system 100 according to an embodiment of the present disclosure may decide ranking among a plurality of users based on at least one of the calculated ranking and exercise points.

FIG. 11 is a diagram illustrating an example of a feedback voice in accordance with still another exemplary embodiment of the present invention.

As illustrated in the drawing, the user terminal 130 according to an embodiment of the present disclosure suggests to provide a feedback voice based on the exercise action judgment.

For example, ranking is decided based on the exercise action judgment of the user, and when the determined ranking is high, feedback of praise 1101-1 can be output to the user 190, and when the determined ranking is low, feedback of encouragement 1101-2 can be output to the user 190.

In an embodiment of the present disclosure, feedback of encouragement 1101-3 can be output to the user 190 according to whether or not a target number of sets is approached based on the number of sets of exercise currently in progress. For example, approximately 10 sets are the target number of sets, but when performing approximately 8 to 10 sets, feedback that the goal is almost reached may be provided to stimulate the user to participate in the exercise until the end.

In addition, in an embodiment of the present disclosure, tempo adjustment feedback 1101-4 may be output according to whether or not it is faster or slower than a preset tempo.

The effects of the smart mat, the terminal, the system, and the control method thereof for interactive fitness will be described as follows.

According to at least one of the embodiments of the present disclosure, there is an advantage in that it is possible to provide an optimal interactive fitness system for home training in terms of cost or space.

In addition, according to at least one of the embodiments of the present disclosure, there is an advantage in that it is possible to induce an active participation of a user doing home training.

Although the embodiment of the interactive fitness system according to the present disclosure has been described above, it is described as at least one embodiment, and the technical spirit of the present invention and its configuration and operation are not limited by the described embodiment, and the scope of the technical spirit of the present invention is not restricted/limited by the drawings or the description made with reference to the drawings. In addition, the concepts and embodiments of the invention presented in the present invention can be used by those of ordinary skill in the art to which the present invention belongs as a basis for modifying or designing the structure of the present invention to other structures in order to perform the same purpose of the present invention, and equivalent structures obtained by modifying or changing the structure of the present invention by those of ordinary skill in the art to which the present invention belongs are bound by the technical scope of the present invention set forth in the claims, and various alterations, substitutions, and changes can be made thereto without departing from the spirit or scope of the invention described in the claims.

Although the smart mat, terminal, system and control method thereof for interactive training have been described with reference to the specific embodiments, they are not limited thereto. Therefore, it will be readily understood by those skilled in the art that various modifications and changes can be made thereto without departing from the spirit and scope of the present invention defined by the appended claims.

1. An interactive fitness terminal, comprising:
a memory configured to store instructions;
a communication unit configured to transmit and receive data with at least one of a smart mat and a wearable terminal; and
a control unit set to execute the stored instructions,
wherein the control unit determines whether or not a user is performing training correctly based on data received through the communication unit.

2. The terminal of claim 1, wherein
the smart mat is configured to acquire contact data, which is data obtained by detecting contact with at least a part of a body of the user.

3. The terminal of claim 2, wherein
the wearable terminal is configured to be worn on a part of the body of the user to acquire motion data, which is data obtained by detecting a motion of the part of the body of the user.

4. The terminal of claim 3, wherein
the control unit is configured to control the communication unit so as to receive at least one of the contact data and the motion data, and make the determination based on at least one of the received contact data and motion data.

5. The terminal of claim 1, wherein
the control unit is configured to output the determined result.

6. A control method of an interactive fitness terminal, comprising:
receiving data from at least one of a smart mat and a wearable terminal; and
determining whether or not a user is performing training correctly based on the received data.

7. The method of claim 6, wherein
the smart mat is configured to acquire contact data, which is data obtained by detecting contact with at least a part of a body of a user.

8. The method of claim 7, wherein
the wearable terminal is configured to be worn on a part of the body of the user to acquire motion data, which is data obtained by detecting a motion of the part of the user's body.

9. The method of claim 8, wherein
in the receiving, at least one of the contact data and the motion data is received, and the determination is made based on at least one of the received contact data and motion data.

10. The method of claim 8, further comprising:
outputting the determined result.

11. A computer program stored on a medium in order to execute the method of claim 6 in combination with hardware.

12. A smart mat, comprising:
a sensing unit configured to detect contact with at least a part of a body of a user;
a communication unit configured to receive motion data from a wearable terminal;
a control unit configured to determine whether or not the user is performing training correctly based on the detected contact and the received motion data; and
an output unit configured to output the determination result.

13. The mat of claim 12, wherein
the motion data is a detection result of at least one of an acceleration sensor and a gyro sensor provided in the wearable terminal.

14. The mat of claim 12, wherein
the output determination result includes a feedback voice.

15. An interactive fitness system, comprising:
a smart mat configured to acquire contact data, which is data obtained by detecting contact with at least a part of a body of a user;
a user terminal configured to output a training video image; and
a server configured to receive motion data acquired through the wearable terminal worn on a part of the body of the user and the contact data,
wherein the server is configured to determine whether or not the user is performing training correctly by combining the motion data and the contact data.
